(19) United States
(12) Patent Application Publication
(10) Pub. No.: US 2022/0370029 A1
(43) Pub. Date: Nov. 24, 2022

(54) CONTROL DEVICE, MOBILE MEDICAL IMAGING APPARATUS, CONTROL METHOD, AND CONTROL PROGRAM

(71) Applicant: FUJIFILM CORPORATION, Tokyo (JP)

(72) Inventors: Koji TANINAI, Kanagawa (JP); Hiromu HAYASHI, Kanagawa (JP); Kazuhiro MAKINO, Kanagawa (JP); Takeyasu KOBAYASHI, Kanagawa (JP); Akihito BETTOYASHIKI, Kanagawa (JP); Masaaki OKAZAKI, Kanagawa (JP)

(21) Appl. No.: 17/744,770

(22) Filed: May 16, 2022

(30) Foreign Application Priority Data
May 19, 2021 (JP) 2021-084858

(51) Int. Cl. A61B 6/00

(52) U.S. Cl. CPC A61B 6/5205; A61B 6/4405; A61B 6/4441; A61B 6/56

(57) ABSTRACT

A console includes a CPU and a GPU. Of the CPU and GPU, the CPU acquires an image to be processed, which is an object to be subjected to a support process that is a diagnosis support process or an imaging support process, and distributes a process to any one of the CPU, the GPU, or another GPU to execute the support process according to the content of the support process executed for the image to be processed.

### CROSS-REFERENCE TO RELATED APPLICATIONS

The present application claims priority under 35 U.S.C. § 119 to Japanese Patent Application No. 2021-084858 filed on May 19, 2021. The above application is hereby expressly incorporated by reference, in its entirety, into the present application.

### BACKGROUND

### 1. Technical Field

The present disclosure relates to a control device, a mobile medical imaging apparatus, a control method, and a control program.

### 2. Description of the Related Art

A technique is known which executes various support processes for images such as radiographic images. For example, JP2012-5894A discloses a medical image support diagnostic device that has a plurality of diagnosis support algorithms executed for medical images.

### SUMMARY

However, a device is known which comprises a plurality of processors as a processor that executes a support process such as diagnosis support. The plurality of processors include, for example, a graphics processing unit (GPU) that is provided inside the host device and a GPU that is provided inside an external server. For some of the processors that execute the support process, a processing result may not be obtained at the desired time, or the processing time may be long. Therefore, a technique that causes an appropriate processor to execute the support process is desired.

The present disclosure has been made in view of the above circumstances, and an object of the present disclosure is to provide a control device, a mobile medical imaging apparatus, a control method, and a control program that can cause an appropriate processor to execute a support process for an image to be processed.

In order to achieve the above object, according to a first aspect of the present disclosure, there is provided a control device comprising two or more processors. One of the two or more processors acquires an image to be processed which is an object to be subjected to a support process that is a diagnosis support process or an imaging support process, and distributes a process to one of a plurality of processors to execute the support process according to content of the support process executed for the image to be processed.

According to a second aspect of the present disclosure, in the control device according to the first aspect, the plurality of processors may include the two or more processors.

According to a third aspect of the present disclosure, in the control device according to the first aspect, the plurality of processors may include an internal processor that is provided inside the control device and an external processor that is provided in a device outside the control device.

According to a fourth aspect of the present disclosure, in the control device according to the third aspect, in a case in which the support process is the imaging support process, the one processor may distribute the process to the internal processor to execute the support process. In a case in which the support process is the diagnosis support process, the one processor may distribute the process to the external processor to execute the support process.

According to a fifth aspect of the present disclosure, in the control device according to any one of the first to third aspects, the one processor may distribute the process to a processor specified from the plurality of processors to execute the support process according to a real-time property of the support process.

According to a sixth aspect of the present disclosure, in the control device according to any one of the first to third aspects, the one processor may distribute the process to a processor specified from the plurality of processors to execute the support process according to a processing load caused by the execution of the support process.

According to a seventh aspect of the present disclosure, in the control device according to any one of the first to sixth aspects, the one processor may control a time when the plurality of processors execute the support process according to the content of the support process executed for the image to be processed.

According to an eighth aspect of the present disclosure, in the control device according to any one of the first to seventh aspects, the one processor may acquire the image to be processed for each process in a series of a plurality of processes from capture of a medical image to a diagnosis of the medical image and distribute the process to one of the plurality of processors to execute the support process for each of the images to be processed.

According to a ninth aspect of the present disclosure, in the control device according to any one of the first to eighth aspects, the one processor may acquire the image to be processed from each of a plurality of imaging apparatuses and distribute the process to a processor specified from the plurality of processors to execute the support process according to the content of the support process for each of the acquired images to be processed.

According to a tenth aspect of the present disclosure, in the control device according to any one of the first to ninth aspects, the plurality of processors may differ from each other in at least one of a communication environment with the control device or a performance.

In order to achieve the above object, according to an eleventh aspect of the present disclosure, there is provided a mobile medical imaging apparatus comprising: the control device according to the present disclosure; and a power source that supplies power to the processors of the control device.

Further, in order to achieve the above object, according to a twelfth aspect of the present disclosure, there is provided a control method comprising: causing one of two or more processors included in a control device to acquire an image to be processed which is an object to be subjected to a support process that is a diagnosis support process or an imaging support process; and causing the one processor to distribute a process to one of a plurality of processors to execute the support process according to content of the support process executed for the image to be processed.

Furthermore, in order to achieve the above object, according to a thirteenth aspect of the present disclosure, there is provided a control program that causes one of two or more processors included in a control device to execute: acquiring an image to be processed which is an object to be subjected to a support process that is a diagnosis support process or an imaging support process; and distributing a process to one of a plurality of processors to execute the support process according to content of the support process executed for the image to be processed.

According to the present disclosure, it is possible to cause an appropriate processor to execute a support process for an image to be processed.

### BRIEF DESCRIPTION OF THE DRAWINGS

Exemplary embodiments according to the technique of the present disclosure will be described in detail based on the following figures, wherein:

FIG. 1 is a diagram illustrating an example of the overall configuration of a radiography system comprising a mobile radiography apparatus and an image processing device according to an embodiment,

FIG. 2 is a block diagram illustrating an example of the configuration of the mobile radiography apparatus and the image processing device according to the embodiment,

FIG. 3 is a functional block diagram illustrating an example of a configuration related to a function of distributing a support process in a console according to the embodiment,

FIG. 4 is a diagram illustrating an example of distribution information,

FIG. 5 is a flowchart illustrating an example of the flow of a distribution process of the console according to the embodiment, and

FIG. 6 is a flowchart illustrating another example of the flow of the distribution process of the console according to the embodiment.

### DETAILED DESCRIPTION

Hereinafter, an embodiment of the present disclosure will be described in detail with reference to the drawings. In the following embodiment, an aspect in which a mobile radiography apparatus comprising a C-arm is applied as an example of a mobile medical imaging apparatus according to the present disclosure will be described. In addition, this embodiment does not limit the present disclosure.

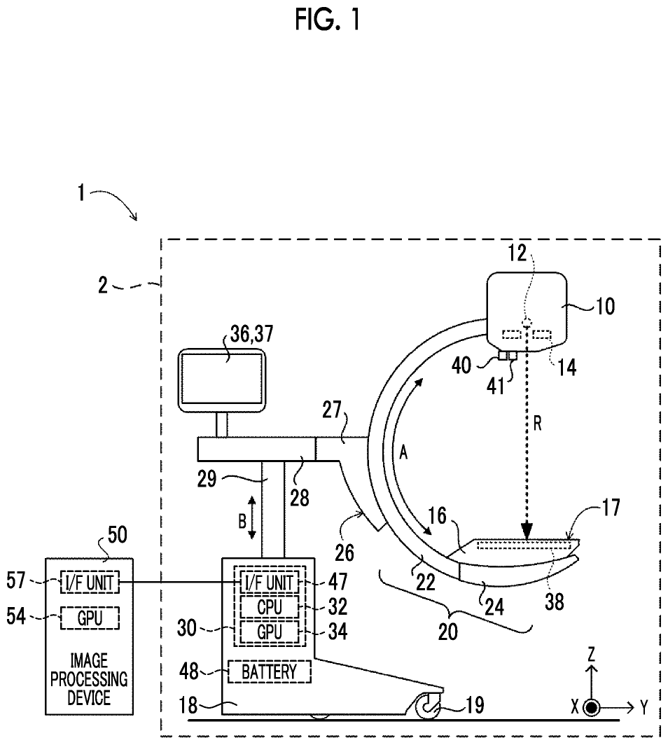

First, the configuration of the mobile radiography apparatus according to this embodiment will be described. FIG. 1 is a diagram illustrating an example of the overall configuration of a radiography system 1 comprising a mobile radiography apparatus 2 and an image processing device 50 according to this embodiment. As illustrated in FIG. 1, the radiography system 1 according to this embodiment comprises the mobile radiography apparatus 2 that captures a radiographic image of a subject and the image processing device 50 that executes a support process for an image.

As illustrated in FIG. 1, the mobile radiography apparatus 2 according to this embodiment comprises a C-arm 20 having an arm portion 22 and a holding portion 24. A radiation emitting unit 10 that emits radiation R generated by a radiation source 12 is provided at one end of the arm portion 22.

The radiation source 12 and an irradiation field limiter 14 are accommodated in the radiation emitting unit 10. The radiation source 12 has a radiation tube (not illustrated) that generates the radiation R and has a function of emitting the radiation R generated by the radiation tube. The irradiation field limiter 14 is a so-called collimator that has a function of limiting the irradiation field of the radiation R generated by the radiation tube. For example, the irradiation field limiter 14 has a configuration in which four shielding plates made of lead or the like that shields the radiation R are disposed on each side of a quadrangle and a quadrangular opening portion for transmitting the radiation R is formed in a central portion. The irradiation field limiter 14 changes the position of each shielding plate to change the size of the opening portion, thereby changing the irradiation field of the radiation R.

Further, as illustrated in FIG. 1, a visible light camera 40 and a depth camera 41 are provided on the side where the radiation R is emitted from the radiation emitting unit 10, that is, on the side facing a radiation detector 38. The visible light camera 40 is a visible light imaging device that captures a visible light image. In this embodiment, the visible light camera 40 can capture at least a moving image. Specifically, the visible light camera 40 has a function of capturing a still image at a predetermined interval and sequentially outputting image data indicating the captured still image as a moving image. In addition, in this embodiment, the “moving image” means a set of still images that are continuous in time. Further, in this embodiment, one frame of a still image that is the source of the moving image is referred to as a “frame”.

Furthermore, the depth camera 41 is a camera that captures a distance image indicating the distance to an object to be imaged. An example of the depth camera 41 is a camera that captures a distance image using a time-of-flight (TOF) method. Specifically, the depth camera 41 irradiates the object to be imaged with light, such as infrared rays, and measures the distance between the depth camera 41 and the object to be imaged, specifically, the distance between the depth camera 41 and a surface of the object to be imaged, on the basis of the time until light reflected from the object to be imaged is received or a change in phase between the emitted light and the received light. In addition, the depth camera 41 does not measure the distance to an object to be imaged which is present behind (shadow) another object to be imaged as viewed from the depth camera 41 among the objects to be imaged which are present in an imaging region.

In the distance image captured by the depth camera 41, each pixel has distance information indicating the distance between the depth camera 41 and the object to be imaged. The distance image captured by the depth camera 41 according to this embodiment has information indicating the distance between the depth camera 41 and the object to be imaged as the pixel value of each pixel. Further, in this embodiment, the distance image means an image from which the distance to the object to be imaged can be derived. Furthermore, the distance image may be either a moving image or a still image. For example, the depth camera 41 according to this embodiment can capture at least the distance image which is a moving image.

In this embodiment, the operator uses the visible light image captured by the visible light camera 40 and the distance image captured by the depth camera 41 to check the positioning of the subject. Therefore, the range required for checking the positioning of the subject whose image is to be captured by the radiation detector 38 is the imaging range of the visible light camera 40 and the imaging range of the depth camera 41.

On the other hand, the holding portion 24 is provided at the other end of the arm portion 22. The holding portion 24 holds an accommodation portion 16. The accommodation portion 16 accommodates the radiation detector 38 that detects the radiation R and generates image data indicating a radiographic image. The C-arm 20 according to this embodiment has a function of changing the angle of the radiation detector 38 with respect to the Z direction (vertical direction) illustrated in FIG. 1.

The radiation detector 38 detects the radiation R transmitted through the subject. Specifically, the radiation detector 38 detects the radiation R that has entered the accommodation portion 16 and reached a detection surface of the radiation detector 38, generates a radiographic image on the basis of the detected radiation R, and outputs image data indicating the generated radiographic image. In the following description, in some cases, a series of operations of emitting the radiation R from the radiation source 12 and generating a radiographic image using the radiation detector 38 is referred to as “imaging”. The type of the radiation detector 38 according to this embodiment is not particularly limited. For example, the radiation detector 38 may be an indirect-conversion-type radiation detector that converts the radiation R into light and converts the converted light into charge or a direct-conversion-type radiation detector that directly converts the radiation R into charge. Further, in this embodiment, the radiation detector 38 can capture a still image and a moving image. In addition, the radiographic image captured as a moving image is also called a fluoroscopic image.

An imaging surface 17 irradiated with the radiation R emitted from the radiation emitting unit 10 is provided on a side of the accommodation portion 16 which faces the radiation emitting unit 10. In addition, in the mobile radiography apparatus 2 according to this embodiment, a so-called source to image distance (SID) which is a distance between the imaging surface 17 and the radiation source 12 of the radiation emitting unit 10 is a fixed value.

The C-arm 20 is held by a C-arm holding portion 26 so as to be movable in the direction of an arrow A illustrated in FIG. 1. Further, the C-arm holding portion 26 has a shaft portion 27, and the shaft portion 27 connects the C-arm 20 to a bearing 28. The C-arm 20 is rotatable about the shaft portion 27 as a rotation axis.

Furthermore, as illustrated in FIG. 1, the mobile radiography apparatus 2 according to this embodiment comprises a main body portion 18 that has a plurality of wheels 19 provided at the bottom. A support shaft 29 that is expanded and contracted in the Z-axis direction of FIG. 1 is provided in an upper part of a housing of the main body portion 18 in FIG. 1. The bearing 28 is held in the upper part of the support shaft 29 so as to be movable in the direction of an arrow B.

Further, a display unit 36 and an operation unit 37 are provided in the upper part of the main body portion 18. The display unit 36 and the operation unit 37 function as a user interface. The display unit 36 provides an operator, such as a technician or a doctor, who takes a radiographic image with the mobile radiography apparatus 2 with the captured radiographic image or information related to the capture of the radiographic image. The display unit 36 is not particularly limited. Examples of the display unit 36 include a liquid crystal monitor and a light emission diode (LED) monitor. In addition, in this embodiment, a touch panel display integrated with the operation unit 37 is applied as an example of the display unit 36. Further, the operator operates the operation unit 37 to input an instruction related to the capture of a radiographic image. The operation unit 37 is not particularly limited. Examples of the operation unit 37 include various switches, a touch panel, a touch pen, and a mouse. Furthermore, a plurality of operation units 37 may be provided. For example, a touch panel and a foot switch operated by the operator with his or her feet may be provided as the operation unit 37.

Moreover, the main body portion 18 accommodates, for example, a central processing unit (CPU) 32, a graphics processing unit (GPU) 34, and an interface (I/F) unit 47 of a console 30 and a battery 48 that supplies power to the mobile radiography apparatus 2.

The console 30 transmits and receives various kinds of information to and from an I/F unit 57 of the image processing device 50 through the I/F unit 47, using wireless communication or wired communication. The image processing device 50 comprises a GPU 54 that performs the support process which will be described in detail below.

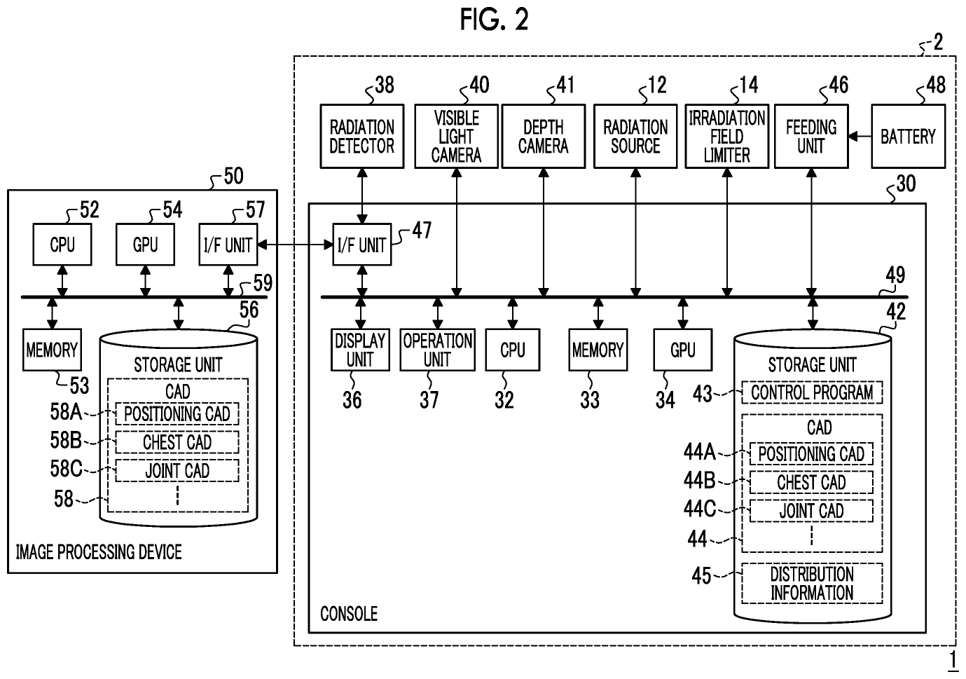

FIG. 2 is a block diagram illustrating an example of the configuration of the mobile radiography apparatus 2 and the image processing device 50 according to this embodiment. As illustrated in FIG. 2, the mobile radiography apparatus 2 according to this embodiment comprises the radiation source 12, the irradiation field limiter 14, the console 30, the radiation detector 38, the visible light camera 40, the depth camera 41, a feeding unit 46, and the battery 48.

The console 30 has a function of performing control related to the capture of a radiographic image by the mobile radiography apparatus 2. In addition, the console 30 according to this embodiment has a function of distributing a process to any one of the CPU 32 of the console 30, the GPU 34 of the console 30, or the GPU 54 of the image processing device 50 according to the content of the support process for the radiographic image, the visible light image, or the distance image. The CPU 32, the GPU 34, and the GPU 54 differ from each other in at least one of a communication environment with the console 30 or the performance, which will be described in detail below. Further, the console 30 according to this embodiment is an example of a control device according to the present disclosure.

In addition, there are a plurality of types of support processes according to this embodiment depending on the content. From the viewpoint of supporting the operator, the types of support processes mainly include an imaging support process and a diagnosis support process. The imaging support process is a process that is performed on an image in order to support the capture of the radiographic image of the subject by the mobile radiography apparatus 2. A plurality of types of processes are given as examples of the imaging support process according to, for example, a method for supporting imaging and the part of the subject to be imaged. An example of the imaging support process is a positioning support process that is performed for the visible light image captured by the visible light camera 40 in order to support the positioning of the subject by the operator. Further, an example of the imaging support process is a positioning support process that is performed on the distance image captured by the depth camera 41 in order to support the positioning of the subject by the operator. On the other hand, the diagnosis support process is a process that is performed for the radiographic image in order to support the interpretation of the radiographic image captured by the mobile radiography apparatus 2. In addition, examples of the diagnosis support process include a diagnosis support process for supporting the interpretation of the position or state of a surgical tool or the like in the radiographic image, a diagnosis support process corresponding to the part to be imaged or the like, and a diagnosis support process for supporting the interpretation of various lesions or the like. As described above, the types of the imaging support process and the diagnosis support process are not particularly limited. For example, a plurality of types of support processes can be provided depending on the operator, the interpreter, the subject, the imaging method, and the like.

The console 30 comprises the CPU 32, a memory 33, the GPU 34, the display unit 36, the operation unit 37, a storage unit 42, and the I/F unit 47. The CPU 32, the memory 33, the GPU 34, the display unit 36, the operation unit 37, the storage unit 42, and the I/F unit 47 are connected to each other through a bus 49, such as a system bus or a control bus, such that they can transmit and receive various kinds of information. In addition, the radiation source 12, the irradiation field limiter 14, the visible light camera 40, the depth camera 41, and the feeding unit 46 are also connected to the bus 49.

The CPU 32 reads out various programs including a control program 43 stored in the storage unit 42 to the memory 33 and executes a process corresponding to the read-out program. Therefore, the CPU 32 controls the operation of each unit of the mobile radiography apparatus 2. Further, the control program 43 according to this embodiment is composed of a plurality of programs including a program for executing a distribution process which will be described below. The memory 33 is a work memory that is used by the CPU 32 to perform processes. The GPU 34 has a function of applying a computer-assisted detection/diagnosis (CAD) 44 stored in the storage unit 42 to execute the support process, which will be described in detail below, under the control of the CPU 32.

The storage unit 42 stores, for example, the control program 43, the CAD 44 which is an algorithm applied to the support process, distribution information 45 which will be described in detail below, the image data of the radiographic image captured by the radiation detector 38, and various other kinds of information. The CAD 44 includes various algorithms corresponding to the types of support processes which will be described below. For example, the CAD 44 according to this embodiment includes a positioning CAD 44A that is applied to the imaging support process for supporting positioning. The details of the positioning CAD 44A are not particularly limited as long as the positioning CAD 44A can provide information for supporting the positioning required in the capture of a fluoroscopic image by the mobile radiography apparatus 2. For example, the positioning CAD 44A may be a CAD for determining whether or not a desired part of the subject is located in the irradiation field. Further, for example, whether the subject lies prone or supine, that is, the orientation of the subject with respect to the radiation detector 38 may be important as positioning. In this case, the positioning CAD 44A may be used as a CAD for determining whether or not the orientation of the subject is appropriate.

In addition, the CAD 44 includes, for example, a chest CAD 44B and a joint CAD 44C which are applied to the diagnosis support process for supporting the interpretation of a radiographic image. The details of each of the chest CAD 44B and the joint CAD 44C are not particularly limited. The chest CAD 44B may be any CAD for supporting interpretation required in a case in which the part to be imaged is the chest. For example, the chest CAD 44B may be at least one of a CAD that specifies the position of the tip of a catheter in a case in which the catheter is inserted or a CAD that specifies a lesion such as pneumothorax or cardiac hypertrophy. Further, the joint CAD 44C may be any CAD that supports interpretation required in a case in which the part to be imaged is a joint. For example, the joint CAD 44C may be at least one of a CAD that specifies a fracture or a CAD that specifies the degree of bending of a bone.

In addition, the algorithms included in the CAD 44 are not limited thereto. A specific example of the storage unit 42 is a hard disk drive (HDD), a solid state drive (SSD), or the like.

The I/F unit 47 transmits and receives various kinds of information to and from the radiation detector 38 using wireless communication or wired communication. Further, the I/F unit 47 transmits and receives various kinds of information to and from the image processing device 50 or other external devices through a network using wireless communication or wired communication. Other external devices include, for example, a radiology information system (RIS) that manages an imaging order and a picture archiving and communication system (PACS).

Furthermore, as described above, the feeding unit 46 is connected to the bus 49. The feeding unit 46 supplies power from the battery 48 to each unit of the mobile radiography apparatus 2. The feeding unit 46 includes, for example, a direct current (DC)-DC converter that converts a direct current voltage from the battery 48 into a voltage having a value corresponding to a supply destination and a voltage stabilizing circuit that stabilizes the value of the converted voltage. The battery 48 according to this embodiment is provided in the main body portion 18 as described above. As described above, the mobile radiography apparatus 2 is wirelessly driven by the battery 48. In addition, a power cord plug (not illustrated) that extends from the bottom of the main body portion 18 in the mobile radiography apparatus 2 can be inserted into an outlet of a commercial power supply to charge the battery 48, or the mobile radiography apparatus 2 can be operated by power from the commercial power supply.

On the other hand, the image processing device 50 has a function of executing the support process, which is the diagnosis support process or the imaging support process, for the image under the control of the console 30. As illustrated in FIG. 2, the image processing device 50 comprises a CPU 52, a memory 53, the GPU 54, a storage unit 56, and the I/F unit 57. The CPU 52, the memory 53, the GPU 54, the storage unit 56, and the I/F unit 57 are connected to each other through a bus 59, such as a system bus or a control bus, such that they can transmit and receive various kinds of information.

The CPU 52 reads out various programs stored in the storage unit 56 to the memory 53 and executes processes according to the read-out programs. Therefore, the CPU 52 controls the operation of each unit of the image processing device 50. The memory 53 is a work memory that is used by the CPU 52 to perform processes. The GPU 54 has a function of applying a CAD 58 stored in the storage unit 56 to execute the support process which will be described in detail below under the control of the CPU 52.

The storage unit 56 stores, for example, various programs (not illustrated) executed by the CPU 52, the CAD 58 which is an algorithm applied to the support process, and various other kinds of information. The CAD 58 includes various algorithms corresponding to the type of the support process which will be described below. For example, the CAD 58 according to this embodiment includes a positioning CAD 58A that is applied to the imaging support process for supporting positioning. Further, the CAD 58 includes, for example, a chest CAD 58B and a joint CAD 58C that are applied to the diagnosis support process for supporting the interpretation of a radiographic image. In addition, the algorithms included in the CAD 58 are not limited thereto. A specific example of the storage unit 56 is an HDD or an SSD. The OF unit 57 transmits and receives various kinds of information to and from the console 30 of the mobile radiography apparatus 2 using wireless communication or wired communication.

In addition, in this embodiment, the CPU 32 and the GPU 34 of the console 30 and the GPU 54 of the image processing device 50 are an example of two or more processors according to the present disclosure. Further, the CPU 32 according to this embodiment is an example of one of the two or more processors according to the present disclosure. Furthermore, the CPU 32 and the GPU 34 according to this embodiment are examples of an internal processor according to the present disclosure. Moreover, the GPU 54 according to this embodiment is an example of an external processor according to the present disclosure, and the image processing device 50 according to this embodiment is an example of a device which comprises the external processor according to the present disclosure and is outside the control device.

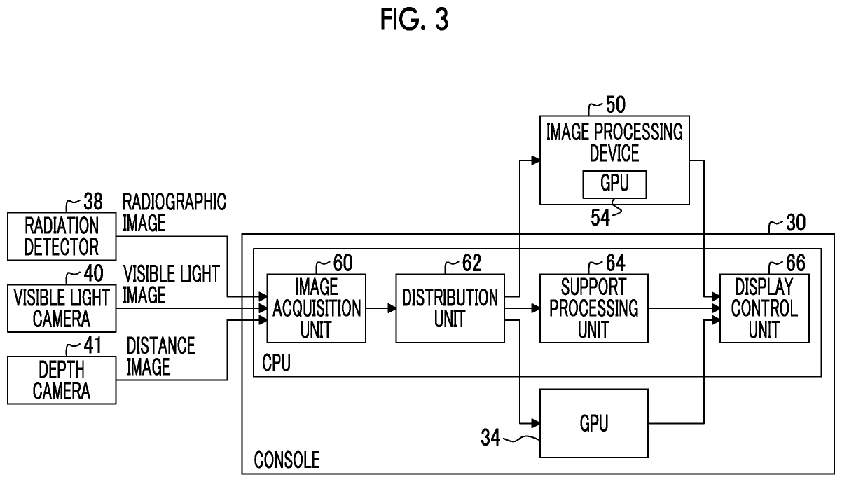

Next, the function of distributing the support process in the console 30 according to this embodiment will be described. FIG. 3 is a functional block diagram illustrating an example of a configuration related to the function of distributing the support process in the console 30 according to this embodiment. As illustrated in FIG. 3, the console 30 comprises an image acquisition unit 60, a distribution unit 62, a support processing unit 64, and a display control unit 66. For example, in the console 30 according to this embodiment, the CPU 32 executes the control program 43 stored in the storage unit 42 to function as the image acquisition unit 60, the distribution unit 62, the support processing unit 64, and the display control unit 66.

The image acquisition unit 60 has a function of acquiring the image to be subjected to the support process which is the diagnosis support process or the imaging support process. Specifically, the image acquisition unit 60 has a function of acquiring the radiographic image captured by the radiation detector 38, the visible light image captured by the visible light camera 40, and the distance image captured by the depth camera 41. Further, in this embodiment, in a case in which the radiographic image, the visible light image, and the distance image which are the images to be subjected to the support process are generically referred to without being distinguished from each other, they are referred to as “images to be processed”. The image acquisition unit 60 outputs image data indicating the acquired images to be processed to the distribution unit 62.

The distribution unit 62 has a function of distributing a process to any one of the support processing unit 64 (CPU 32), the GPU 34, or the GPU 54 so as to execute the support process, according to the content of the support process performed for the images to be processed. Further, in the following description, in a case in which the CPU 32, the GPU 34, and the GPU 54 that execute the support process are generically referred to without being distinguished from each other, they may be simply referred to as “processors”.

The real-time property of the support process and a processing load caused by the execution of the support process are determined according to the content of the support process. Therefore, for example, the distribution unit 62 according to this embodiment distributes the process to one of a plurality of processors so as to execute the support process according to the real-time property of the support process and the processing load caused by the execution of the support process. In addition, the real-time property of the support process means the property that the support process can be ended within a predetermined period after the console 30 acquires the images to be processed. Further, criteria for determining the magnitude of the processing load may be predetermined according to, for example, the image processing capability of each processor or may be set by the operator.

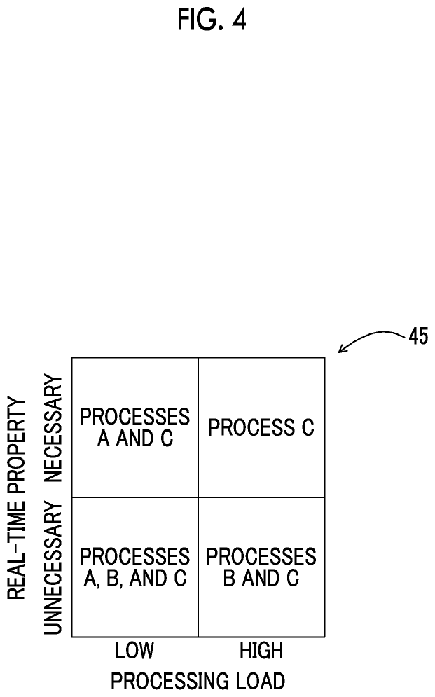

In this embodiment, a correspondence relationship between processes A to C executed in each of the CPU 32, the GPU 34, and the GPU 54, and the real-time property and the processing load is stored as the distribution information 45 in the storage unit 42. FIG. 4 illustrates an example of the distribution information 45.

The process A is a support process executed by the CPU 32 in the console 30, that is, the support processing unit 64. In this embodiment, the image processing capability of the CPU 32 is lower than that of the GPU 34, the GPU 54, and the like. Therefore, it is preferable that the CPU 32 performs a process with a relatively low processing load. Further, since the CPU 32 functioning as the image acquisition unit 60 acquires the images to be processed as described above, the CPU 32 functioning as the support processing unit 64 can quickly execute the support process for the acquired images to be processed. Therefore, the CPU 32 can respond to both a process that requires the real-time property and a process that does not require the real-time property. Therefore, the process A may be the support process having a low processing load, and the real-time property may or may not be necessary.

Examples of the process A include a positioning support process for the visible light image captured by the visible light camera 40 and a positioning support process for the distance image captured by the depth camera 41. Further, a support process for the image to be processed which has a small size is given as an example of the process A. Furthermore, a support process whose frequency is relatively low is given as an example of the process A. In addition, the criteria for the frequency of the support process are not particularly limited. However, for example, the frequency may be adopted on the basis of the frame rate at which the fluoroscopic image is captured by the radiation detector 38.

The process B is a support process that is executed by the GPU 54 of the image processing device 50, specifically, the CPU 52 and the GPU 54 of the image processing device 50. In this embodiment, the GPU 54 has a higher image processing capability than at least the CPU 32 of the console 30. Therefore, the GPU 54 can respond to both a process with a low processing load and a process with a high processing load. Further, as described above, since the image processing device 50 performs wireless communication or wired communication through the I/F unit 57 and the I/F unit 47 of the console 30, it takes time to transmit and receive the image to be processed and the processing result between the console 30 and the image processing device 50.

Therefore, the GPU 54 is not suitable for the process requiring the real-time property. Therefore, the process B may be any process that does not require the real-time property, and the processing load may be high or low.

An example of the process B is a lesion diagnosis support process for one radiographic image.

The process C is a support process that is executed by the GPU 34 in the console 30. In this embodiment, the GPU 34 has a higher image processing capability than at least the CPU 32. Therefore, the GPU 34 can respond to both the process with a low processing load and the process with a high processing load. Further, as described above, the GPU 34 is provided in the console 30 together with the CPU 32. In this embodiment, the GPU 34 is connected to the CPU 32 by the bus 49. Therefore, the GPU 34 can respond to both the process that requires the real-time property and processing that does not require the real-time property. Therefore, the process C may or may not require the real-time property and may have a high processing load or a low processing load.

An example of the process C is a support process for a moving image having a high frame rate such as a fluoroscopic image.

As illustrated in FIG. 4, a support process that requires the real-time property and has a high processing load corresponds to the process C. That is, the distribution unit 62 distributes the support process that requires the real-time property and has a high processing load to the GPU 34 of the console 30. Examples of this support process include a diagnosis support process for a high frame rate moving image, such as a fluoroscopic image, and a diagnosis support process for specifying the position of a surgical tool, such as a catheter, during surgery.

Further, as illustrated in FIG. 4, a support process that requires the real-time property and has a low processing load corresponds to the process A and the process C. That is, the distribution unit 62 distributes the support process that requires the real-time property and has a low processing load to the CPU 32 or the GPU 34 of the console 30. An example of this support process is a positioning support process for supporting the positioning that needs to be determined in real time. For example, a positioning support process for determining whether or not a desired part of the subject is located in the irradiation field is given as an example of the support process.

Furthermore, as illustrated in FIG. 4, a support process that does not require the real-time property and has a high processing load corresponds to the process B or the process C. That is, the distribution unit 62 distributes the support process that does not require the real-time property and has a high processing load to the GPU 54 of the image processing device 50 or the GPU 34 of the console 30. An example of this support process is a lesion diagnosis support process for a radiographic image.

Moreover, as illustrated in FIG. 4, a support process that does not require the real-time property and has a low processing load corresponds to the processes A to C. That is, the distribution unit 62 distributes the support process that does not require the real-time property and has a low processing load to the CPU 32 or the GPU 34 of the console 30 or the GPU 54. Examples of this support process include a support process for the visible light image captured by the visible light camera 40 or the distance image captured by the depth camera 41 and a support process for the image to be processed which has a small size.

Therefore, the distribution unit 62 according to this embodiment specifies whether the processor which is the distribution destination for executing the support process is the support processing unit 64 (CPU 32), the GPU 34, or the GPU 54 on the basis of the distribution information 45. Further, the distribution unit 62 outputs the image to be processed to the specified processor which is the distribution destination and instructs the processor to execute the support process.

In addition, the distribution information 45 is not limited to that illustrated in FIG. 4. For example, the imaging support process has a relatively low processing load. On the other hand, the diagnosis support process has a relatively high processing load. Therefore, the distribution information 45 may be used which instructs the CPU 32 (process A) or the GPU 34 (process C) which is the internal processor of the console 30 to execute the imaging support process and instructs the GPU 54 (process B) of the image processing device 50 which is the external processor of the console 30 to execute the diagnosis support process.

The support processing unit 64 has a function of performing the instructed support process for the image to be processed in a case in which the distribution unit 62 has distributed the support process. Specifically, the support processing unit 64 applies the CAD algorithm selected from the CAD 44 stored in the storage unit 42 to the image to be processed according to the instructed support process to execute the support process. The support processing unit 64 outputs the processing result of the support process to the display control unit 66.

In addition, the GPU 34 also has a function of executing the instructed support process for the image to be processed in a case in which the distribution unit 62 has distributed the support process. Specifically, the GPU 34 applies the CAD algorithm selected from the CAD 44 stored in the storage unit 42 to the image to be processed according to the instructed support process to execute the support process. The GPU 34 outputs the processing result of the support process to the display control unit 66.

Further, the GPU 54 of the image processing device 50 also has a function of executing the instructed support process for the image to be processed in a case in which the distribution unit 62 has distributed the support process. Specifically, the GPU 54 applies the CAD algorithm selected from the CAD 58 stored in the storage unit 56 to the image to be processed, which has been received from the console 30, according to the instructed support process to execute the support process. The GPU 54 outputs the processing result of the support process to the display control unit 66.

The display control unit 66 has a function of displaying the processing result of the support processing unit 64, the processing result of the GPU 34, or the processing result of the GPU 54 on the display unit 36.

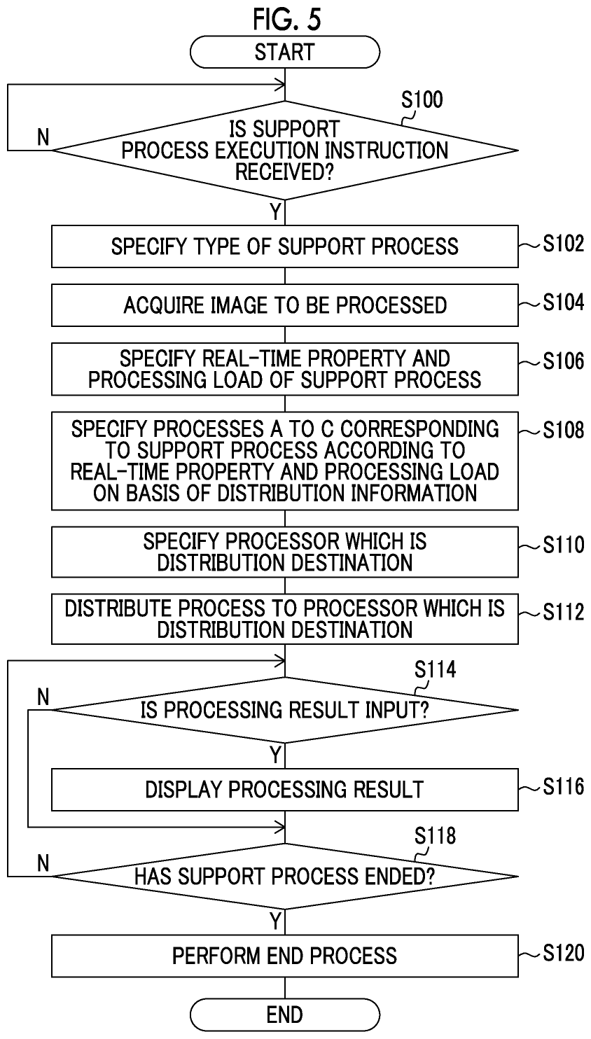

Next, the operation of the console 30 related to the distribution of the support process will be described with reference to the drawings. In a case in which an instruction to capture a radiographic image corresponding to an imaging order is input, the console 30 according to this embodiment executes the distribution process illustrated in FIG. 5. FIG. 5 is a flowchart illustrating an example of the flow of the distribution process executed by the console 30 according to this embodiment. In the console 30 according to this embodiment, for example, the CPU 32 executes the control program 43 stored in the storage unit 42 to perform the distribution process whose example is illustrated in FIG. 4.

In Step S100 of FIG. 5, the image acquisition unit 60 determines whether or not a support process execution instruction has been received. For example, in the console 30 according to this embodiment, in a case in which the image acquisition unit 60 receives the support process execution instruction, the execution of the support process is started. An example of the support process execution instruction is an execution instruction input from the operator through the operation unit 37. Further, an example of the support process execution instruction is an execution instruction that is automatically input at a predetermined time such as the time when the radiation detector 38 captures a radiographic image.

The determination result in Step S100 is “No” until the support process execution instruction is received. On the other hand, in a case in which the support process execution instruction has been received, the determination result in Step S100 is “Yes”, and the process proceeds to Step S102.

In Step S102, the image acquisition unit 60 specifies the type of support process. As described above, in this embodiment, the types of support processes include the imaging support process and the diagnosis support process. Further, there are a plurality of types of imaging support processes and a plurality of types of diagnosis support processes. Therefore, the image acquisition unit 60 specifies the type of support process corresponding to the execution instruction. In addition, the method by which the image acquisition unit 60 specifies the type of support process is not particularly limited. For example, information indicating the type of support process may be associated with the execution instruction received by the image acquisition unit 60. Further, for example, the image acquisition unit 60 may display, on the display unit 36, information indicating the type of support processes, such as the name of the CAD applied in the support process, receive information indicating the type of support process selected by the operator from the displayed information, and specify the type of support process. Furthermore, for example, the type of support process to be executed may be associated with the imaging order, and the image acquisition unit 60 may specify the type of support process associated with the imaging order.

Then, in Step S104, the image acquisition unit 60 acquires the image to be processed. Specifically, the image acquisition unit 60 acquires the image to be processed from any one of the radiation detector 38, the visible light camera 40, or the depth camera 41 according to the type of support process specified in Step S102. In addition, in a case in which the image to be processed is a moving image, the acquisition of the image to be processed is started in this step.

Then, in Step S106, the distribution unit 62 specifies the real-time property and the processing load of the support process corresponding to the execution instruction. In addition, the method by which the distribution unit 62 specifies the real-time property and the processing load of the support process is not particularly limited. For example, the real-time property and the processing load may be predetermined for each type of support process, and the distribution unit 62 may specify the real-time property and the processing load corresponding to the type of support process specified in Step S102. Further, for example, the processing load may be specified according to the size of the image to be processed which has been acquired in Step S104.

Then, in Step S108, the distribution unit 62 specifies which of the processes A to C the support process corresponding to the execution instruction corresponds to, according to the real-time property and the processing load specified in Step S106, on the basis of the distribution information 45 stored in the storage unit 42. As described above, for example, in a case in which the support process corresponding to the execution instruction is the lesion diagnosis support process for the radiographic image acquired from the radiation detector 38, the distribution unit 62 specifies that the support process corresponds to the process B and the process C, with reference to the distribution information 45.

Then, in Step S110, the distribution unit 62 specifies the processor which is the distribution destination of the process. As described above, the processors that execute the processes A to C are determined. Therefore, the distribution unit 62 specifies the processor which is the distribution destination on the basis of the processes A to C specified in Step S108. Specifically, in a case in which the distribution unit 62 specifies that the support process corresponds to the process A, it specifies the CPU 32 as the processor which is the distribution destination. Further, in a case in which the distribution unit 62 specifies that the support process corresponds to the process B, it specifies the GPU 54 of the image processing device 50 as the processor which is the distribution destination. Furthermore, in a case in which the distribution unit 62 specifies that the support process corresponds to the process C, it specifies the GPU 34 as the processor which is the distribution destination. Moreover, in a case in which the support process corresponds to a plurality of processes A to C, for example, in a case in which the support process corresponds to the process B and the process C as described above, there is no particular limitation on which of these processes is used to specify the processor that is the distribution destination. In other words, in a case in which there are a plurality of processors that are candidates for the distribution destination, there is no particular limitation on which of the processors the distribution unit 62 specifies as the distribution destination. For example, the processor that is currently executing the support process may be excluded from the candidates for the distribution destination. Further, for example, priority may be given to the processor to be the distribution destination or the processes A to C in advance, and the distribution unit 62 may specify the distribution destination on the basis of the priority.

Then, in Step S112, the distribution unit 62 distributes the process to the processor, which is the distribution destination specified in Step S110, so as to execute the support process. As described above, the distribution unit 62 according to this embodiment outputs the image to be processed, which has been acquired in Step S104, to the processor which is the distribution destination and instructs the processor to execute the support process.

In a case in which the distribution unit 62 executes the process in this step as described above, the CPU 32 (support processing unit 64) or the GPU 34 of the console 30 or the GPU 54 of the image processing device 50 executes the support process distributed by the distribution unit 62. The CPU 32 (support processing unit 64) or the GPU 34 selects an algorithm corresponding to the support process, which corresponds to the execution instruction, from the CAD 44 stored in the storage unit 42 and applies the selected algorithm to the image to be processed to execute the support process. For example, in a case in which the distribution unit 62 distributes, to the CPU 32 (support processing unit 64), the imaging support process for supporting the positioning which is applied to the visible light image acquired from the visible light camera 40, the CPU 32 selects the positioning CAD 44A from the CAD 44 stored in the storage unit 42. Further, the CPU 32 (support processing unit 64) applies the positioning CAD 44A to the visible light image to execute the imaging support process for supporting positioning.

On the other hand, the GPU 54 selects an algorithm corresponding to the support process, which corresponds to the execution instruction, from the CAD 58 stored in the storage unit 56 and applies the selected algorithm to the image to be processed to execute the support process. For example, in a case in which the distribution unit 62 distributes, to the GPU 54, the diagnosis support process for supporting the interpretation of a lesion which is applied to the radiographic image of the chest as the object to be imaged that has been acquired by the radiation detector 38, the GPU 54 selects the chest CAD 58B from the CAD 58 stored in the storage unit 56. Further, the GPU 54 applies the chest CAD 58B to the radiographic image to execute the diagnosis support process for supporting the interpretation of the lesion.

Then, in Step S114, the display control unit 66 determines whether or not the processing result has been input from the processor which is the distribution destination. The determination result in Step S114 is “No” until the processing result is input, and the process proceeds to Step S118. On the other hand, in a case in which the processing result is input, the determination result in Step S114 is “Yes”, and the process proceeds to Step S116.

In Step S116, the display control unit 66 displays the input processing result on the display unit 36. In addition, the display form of the result of the support process by the display control unit 66 is not particularly limited. For example, the display control unit 66 may display the image to be subjected to the support process on the display unit 36 together with the processing result.

Then, in Step S118, the display control unit 66 determines whether or not the support process has ended. The display control unit 66 according to this embodiment determines that the support process has ended in a case in which predetermined end conditions are satisfied. The end conditions are not particularly limited. For example, in a case in which the image to be processed is a moving image and the support process is continuously executed for a plurality of frames, the end condition may be that a support process end instruction input by the operator through the operation unit 37 has been received. Further, the end condition may be satisfied in the following case: the processor having the process distributed thereto outputs an end signal indicating the end of the support process in a case in which the support process has ended, and the display control unit 66 receives the end signal from the processor which is the distribution destination. The determination result in Step S118 is “No” until the end conditions are satisfied, the process returns to Step S114, and the processes in Steps S114 and S116 are repeated. On the other hand, in a case in which the end conditions are satisfied, the determination result in Step S118 is “Yes”, and the process proceeds to Step S120.

In Step S120, a predetermined end process for ending the support process is executed. For example, in this embodiment, as the end process, the display control unit 66 executes a process of storing the processing result input from the processor which is the distribution destination in a predetermined storage unit such as the storage unit 42. Further, in a case in which the image to be processed is a moving image, the acquisition of the image to be processed is ended. In addition, the end process is not limited to these processes. In a case in which the process in Step S120 ends, the distribution process illustrated in FIG. 5 ends.

As described above, the console 30 according to the above-described embodiment comprises the CPU 32 and the GPU 34. Of the CPU 32 and GPU 34, the CPU 32 acquires the image to be processed, which is the object to be subjected to the support process that is the diagnosis support process or the imaging support process, and distributes the process to any one of the CPU 32, the GPU 34, or the GPU 54 so as to execute the support process according to the content of the support process executed for the image to be processed.

As described above, the console 30 according to the above-described embodiment distributes the process to one of a plurality of processors so as to execute the support process according to the content of the support process. Therefore, for example, according to the console 30 of the above-described embodiment, it is possible to cause the CPU 32 or the GPU 34 in the console 30, which can secure the real-time property, to execute the support process requiring the real-time property, without causing the GPU 54, which is less likely to secure the real-time property, to execute the support process. Further, for example, according to the console 30 of the above-described embodiment, it is possible to cause the GPU 34 or the GPU 54, which has a relatively high image processing capability, to execute the support process having a relatively high processing load, without causing the CPU 32, which has a relatively low image processing capability, to execute the support process. Therefore, according to the console 30 of the above-described embodiment, it is possible to direct an appropriate processor to execute the support process on the image to be processed.

In addition, the distribution process executed for one support process is illustrated with reference to FIG. 5, and the aspect in which the distribution process is executed whenever the support process execution instruction is received has been described above. For this aspect, in a case in which a plurality of support processes are sequentially executed, the processors that execute each support process may be distributed in advance according to the order in which the plurality of support processes are executed. For example, in a series of a plurality of processes from the capture of a medical image to the diagnosis of the medical image, the image to be processed may be acquired for each process, and the process may be distributed to one of a plurality of processors in advance so as to execute the support process for each of the images to be processed.

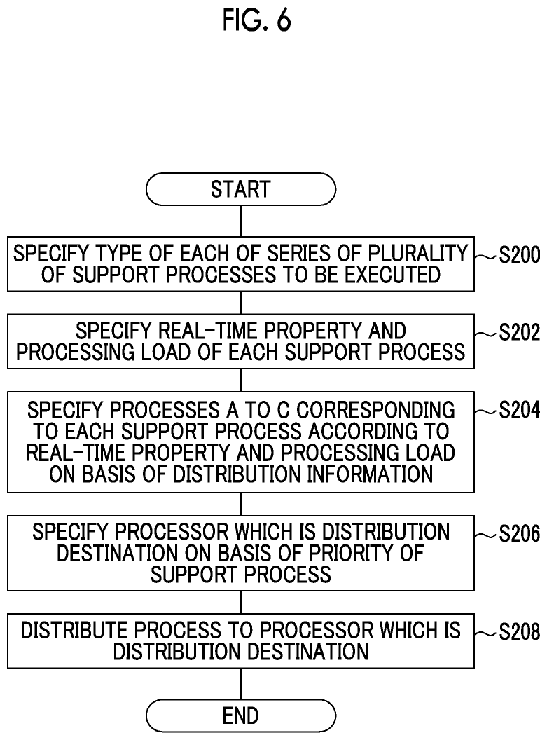

FIG. 6 is a flowchart illustrating an example of the flow of the distribution process in a case in which, in a series of a plurality of processes, the support process to be applied to the image to be processed is distributed in advance for each process. The distribution process illustrated in FIG. 6 is executed, for example, in a case in which the console 30 receives an imaging order.

In Step S200 of FIG. 6, the distribution unit 62 specifies the type of each of a plurality of support processes to be executed. For example, there are the following stages from the capture of the medical image to the diagnosis of the medical image: the “positioning of the subject”; the “setting of an irradiation dose”; the “capture of a radiographic image”; “checking whether or not to perform reimaging”; and the “diagnosis of lesions”. Among these stages, the support processes for the stage in which the support process is performed is referred to as a series of a plurality of processes. For example, the following case is given as an example: for the positioning of the subject, three support processes, that is, a positioning support process for supporting positioning, a support process that inserts a catheter and specifies the position of the tip of the catheter in the capture of a fluoroscopic image, and a diagnosis support process for lesions and the like are executed as a series of a plurality of support processes. The distribution unit 62 specifies the type of each of the plurality of support processes on the basis of, for example, the above-mentioned stages.

Then, in Step S202, the distribution unit 62 specifies the real-time property and the processing load of each support process on the basis of the type of the support process specified in Step S200. The distribution unit 62 specifies the real-time property and the processing load of each support process as in Step S106 of the distribution process illustrated in FIG. 5.

Then, in Step S204, the distribution unit 62 specifies which of the processes A to C each of the plurality of support processes corresponds to, according to the real-time property and the processing load specified in Step S202, on the basis of the distribution information 45 stored in the storage unit 42. The distribution unit 62 specifies which of the processes A to C each support process corresponds to, as in Step S108 of the distribution process illustrated in FIG. 5.

Then, in Step S206, the distribution unit 62 specifies a processor which is the distribution destination on the basis of the priority of the support process. In a case in which a series of a plurality of support processes is executed, the plurality of support processes may be executed at the same time. For example, in a case in which the positioning support process for supporting positioning in order to properly position the subject is executed while the support process that inserts a catheter and specifies the position of the tip of the catheter in the capture of a fluoroscopic image, two support processes are executed at the same time. In this aspect, in a case in which the support processes executed at the same time are distributed, the distribution unit 62 specifies a processor which is the distribution destination on the basis of the priority of the support processes. In addition, the priority of the support processes may be predetermined or may be set by the operator. Further, the processing results may be obtained in the order desired by the operator or the interpreter, and how to specify the processor which is the distribution destination on the basis of the priority of the support processes is not particularly limited. For example, the distribution unit 62 may distribute a support process having a low priority to a processor having a lower image processing capability than a processor to which a support process having a high priority is distributed. Further, the distribution unit 62 may control the time when the processor executes the support process such that a support process having a low priority and a low real-time property is executed after the other support processes end.

Then, in Step S208, the distribution unit 62 distributes the process to the processor which is the distribution destination specified in Step S206 so as to execute the support process. The distribution unit 62 distributes the process to the specified processor which is the distribution destination so as to execute the support process as in Step S112 of the distribution process illustrated in FIG. 5. In a case in which the process in Step S208 ends, the distribution process illustrated in FIG. 6 ends.

In addition, in the distribution process illustrated in FIG. 6, the process is distributed to one of a plurality of processors such that the processor executes the support process before the support process is actually executed. For example, the distribution process is executed in a case in which the console 30 receives an imaging order. Therefore, the console 30 performs the processes in Step S104 and Steps S114 to S120 of the distribution process illustrated in FIG. 5 to direct the processor, which is the distribution destination, to execute the support process at the time when the support process is actually executed and displays the processing result on the display unit 36.

Further, in the above-described embodiment, the aspect in which each of the processors of the support processing unit 64 (CPU 32), the GPU 34, and the GPU 54 applies a CAD algorithm to execute the diagnosis support process has been described. However, the aspect of the support process or the CAD is not limited to this embodiment. For example, each processor may apply artificial intelligence (AI) technology to execute the support process or may apply a trained model, which has been machine-learned by deep learning or the like, to execute the support process.

Furthermore, in the above-described embodiment, the aspect in which the console 30 is an example of the control device according to the present disclosure has been described. However, devices other than the console 30 may have the functions of the control device according to the present disclosure. In other words, a device other than the console 30 may comprise some or all of the image acquisition unit 60, the distribution unit 62, the support processing unit 64, and the display control unit 66.

Moreover, in the above-described embodiment, the aspect in which the CPU 32 and the GPU 34 comprised in the console 30 are connected by the bus 49 has been described. However, the connection mode between the CPU 32 and the GPU 34 is not limited to this embodiment. For example, the GPU 34 and a storage unit that stores various CAD algorithms may be provided as a so-called GPU box separately from the CPU 32, and the CPU 32 and the GPU 34 may be connected by high-speed communication.

In addition, in the above-described embodiment, the aspect in which the mobile radiography apparatus having the C-arm is applied as an example of the mobile medical imaging apparatus has been described. However, the mobile medical imaging apparatus is not limited to this embodiment. For example, a combination of a mobile cart having the radiation emitting unit 10 and the radiation detector 38 which is a so-called electronic cassette may be used as the mobile medical imaging apparatus. Further, for example, a portable medical imaging apparatus that the operator carries and moves may be used. Furthermore, the imaging apparatus is not limited to the mobile medical imaging apparatus and may be a stationary medical imaging apparatus. Moreover, for example, the imaging apparatus may be a medical imaging apparatus that captures a computed tomography (CT) image, an ultrasound image, or the like.

In addition, in the above-described embodiment, for example, the following various processors can be used as a hardware structure of processing units performing various processes, such as the image acquisition unit 60, the distribution unit 62, the support processing unit 64, and the display control unit 66. The various processors include, for example, a CPU which is a general-purpose processor executing software (programs) to function as various processing units as described above, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform a specific process.

One processing unit may be configured by one of the various processors or a combination of two or more processors of the same type or different types (for example, a combination of a plurality of FPGAs or a combination of a CPU and an FPGA). Further, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As such, various processing units are configured by using one or more of the various processors as the hardware structure.

In addition, specifically, an electric circuit (circuitry) obtained by combining circuit elements, such as semiconductor elements, can be used as the hardware structure of the various processors.

In addition, in the above-described embodiment, the aspect in which the control program 43 is stored (installed) in the storage unit 42 in advance has been described. However, the present disclosure is not limited thereto. The control program 43 may be recorded on a recording medium, such as a compact disc read only memory (CD-ROM), a digital versatile disc read only memory (DVD-ROM), or a universal serial bus (USB) memory, and then provided. In addition, the control program 43 may be downloaded from an external device through a network.

What is claimed is:

1. A control device comprising:
two or more processors,
wherein one of the two or more processors acquires an image to be processed which is an object to be subjected to a support process that is a diagnosis support process or an imaging support process, and distributes a process to one of a plurality of processors to execute the support process according to content of the support process executed for the image to be processed.

2. The control device according to claim 1,
wherein the plurality of processors include the two or more processors.

3. The control device according to claim 1,
wherein the plurality of processors include an internal processor that is provided inside the control device and an external processor that is provided in a device outside the control device.

4. The control device according to claim 3,
wherein, in a case in which the support process is the imaging support process, the one processor distributes the process to the internal processor to execute the support process, and
in a case in which the support process is the diagnosis support process, the one processor distributes the process to the external processor to execute the support process.

5. The control device according to claim 1,
wherein the one processor distributes the process to a processor specified from the plurality of processors to execute the support process according to a real-time property of the support process.

6. The control device according to claim 1,
wherein the one processor distributes the process to a processor specified from the plurality of processors to execute the support process according to a processing load caused by the execution of the support process.

7. The control device according to claim 1,
wherein the one processor controls a time when the plurality of processors execute the support process according to the content of the support process executed for the image to be processed.

8. The control device according to claim 1,
wherein the one processor acquires the image to be processed for each process in a series of a plurality of processes from capture of a medical image to a diagnosis of the medical image, and distributes the process to one of the plurality of processors to execute the support process for each of the images to be processed.

9. The control device according to claim 1,
wherein the one processor acquires the image to be processed from each of a plurality of imaging apparatuses, and distributes the process to a processor specified from the plurality of processors to execute the support process according to the content of the support process for each of the acquired images to be processed.

10. The control device according to claim 1,
wherein the plurality of processors differ from each other in at least one of a communication environment with the control device or a performance.

11. A mobile medical imaging apparatus comprising:
the control device according to claim 1; and
a power source that supplies power to the processors of the control device.

12. A control method comprising:
causing one of two or more processors included in a control device to acquire an image to be processed which is an object to be subjected to a support process that is a diagnosis support process or an imaging support process; and
causing the one processor to distribute a process to one of a plurality of processors to execute the support process according to content of the support process executed for the image to be processed.

13. A non-transitory computer-readable storage medium storing a control program that causes one of two or more processors included in a control device to execute:
acquiring an image to be processed which is an object to be subjected to a support process that is a diagnosis support process or an imaging support process; and
distributing a process to one of a plurality of processors to execute the support process according to content of the support process executed for the image to be processed.
